(19) United States
(12) Patent Application Publication
(10) Pub. No.: US 2022/0296834 A1
(43) Pub. Date: Sep. 22, 2022

(54) ANALYSIS OF MUCUS CHARACTERISTICS

(71) Applicant: KONINKLIJKE PHILIPS N.V., Eindhoven (NL)

(72) Inventors: Pascal de Graaf, Eindhoven (NL); Kiran Hamilton J. Dellimore, Eindhoven (NL); Marco Baragona, Eindhoven (NL); Samer Bou Jawde, Cambridge, MA (US)

(21) Appl. No.: 17/667,606

(22) Filed: Feb. 9, 2022

Related U.S. Application Data
(60) Provisional application No. 63/164,301, filed on Mar. 22, 2021.

(51) Int. Cl. A61M 16/00; A61M 16/06

(52) U.S. Cl. CPC A61M 16/026; A61M 16/06; A61M 2016/0027; A61M 2016/0036

(57) ABSTRACT

A system is for analyzing physical characteristics of mucus, in particular during patient respiratory support using a ventilator. A ventilation waveform is sensed and analyzed and an estimate of, or a change in, at least one mucus characteristic can then be determined.

### CROSS-REFERENCE TO RELATED APPLICATIONS

This patent application claims the priority benefit under 35 U.S.C. § 119(e) of U.S. Provisional Application No. 63/164,301, filed on Mar. 22, 2021, the contents of which are herein incorporated by reference.

### FIELD OF THE INVENTION

The invention relates to a method and system for analyzing characteristics of mucus, in particular for use in devising a suitable non-pharmaceutical therapy for loosening or thinning the mucus.

### BACKGROUND OF THE INVENTION

Many patients with chronic respiratory diseases, such as chronic obstructive pulmonary disease (COPD), cystic fibrosis (CF) and non CF-bronchiectasis, experience severe mucus build-up in their airway system. Consequently, clearing their airways from mucus build-up may become more difficult. This may lead to accumulation of bacterial load, which can in turn cause exacerbations.

Various pharmaceutical and non-pharmaceutical methods are typically employed to first loosen and/or thin the mucus prior to expulsion by coughing. Non-pharmaceutical loosening and/or thinning of mucus is usually achieved by manual means (e.g., chest percussion by a respiratory therapist) or semi-automated means (e.g., High Frequency Chest Wall Oscillation (HFCWO) therapy or Oscillating Positive Expiratory Pressure (OPEP) therapy).

A key unmet need in non-pharmaceutical mucus loosening, thinning and clearance remains the optimization of semi-automated therapies, in order to meet patient-specific mucus removal needs, in particular in a domestic setting. This necessitates quantification of not only the amount of mucus build-up (i.e., the presence of mucus accumulation in the airway), but also the distribution of mucus in the airway (e.g., whether it is in the upper or lower airway) and the physical properties of the mucus, such as the mucus viscosity and thickness. Once obtained, this information can be used to personalize semi-automated mucus loosening, thinning and clearance therapy, by adapting the duration and frequency of clearance and device settings (e.g., applied pressure, force, oscillation frequency, etc.).

In domestic settings, characterization and measurement of mucus physical characteristics is challenging due to limitations in collection methods and techniques for analysis of mucus. In addition, collection of mucus samples by patients is burdensome, undesirable (since it is messy and cumbersome to handle) as well as time-consuming.

It would be desirable to overcome these challenges in mucus characterization and measurement in order to be able to realize optimized and personalized semi-automated mucus clearance therapy. In particular, it would be of particular interest to be able to characterize changes in mucus characteristics without requiring a mucus sample to be obtained and analyzed in vitro.

### SUMMARY OF THE INVENTION

The invention is defined by the claims.

According to examples in accordance with an aspect of the invention, there is provided a system for analyzing physical characteristics of mucus during patient respiratory support using a ventilator, comprising:

a sensor arrangement for sensing a ventilation waveform;

a processing unit configured to:
analyze the sensed ventilation waveform over time; and
derive from the analysis an estimate of, or a change in, at least one mucus characteristic.

This system makes use of characteristic features extracted from one or more ventilation waveforms to estimate and/or capture changes in at least one mucus characteristic, which may relate to the physical properties of the mucus itself or the way mucus is distributed in the airway. This information may for example be used to optimize mucus loosening and clearance therapy.

The system is able to detect changes in mucus characteristics in vivo, by utilizing changes in ventilation waveforms (e.g. a power spectral density), in particular in a flow waveform and/or a pressure waveform. The system eliminates the need for burdensome and time consuming mucus sample collection and handling by patients.

By way of example, changes in mucus thickness and viscosity may be tracked, as well as airway locations of mucus build-up in order to support personalization of a mucus clearance therapy, in particular non-pharmaceutical loosening and/or thinning of mucus.

The at least one mucus characteristic may comprise one or more of:

a mucus thickness;

a mucus viscosity; and

a distribution of mucus in the airway of the patient.

The sensed ventilation waveform for example comprises one or more of:

a ventilation pressure; and

a ventilation flow.

The processing unit may be configured to analyze a frequency spectrum content of the ventilation waveform. Changes in frequency content can result from the presence and thickness changes of mucus.

The processing unit is for example configured to analyze the ventilation waveform during an inspiration phase to determine mucus thickness.

This provides a way to detect automatically or semi-automatically the mucus thickness. For example, a larger spectral content of the ventilation waveform is present for higher thickness cases as a result of a larger induced oscillatory flow. These oscillations increase in amplitude and frequency with increasing mucus thickness for a given respiration rate. These oscillations are less prominent in the expiration phase.

The processing unit may be configured to analyze a power spectral density of the ventilation waveform to determine mucus viscosity.

The mucus viscosity influences oscillatory behavior of the ventilation waveform.

The processing unit is for example configured to determine a maximum power spectral density over a number of successive breaths thereby to determine the mucus viscosity.

A higher viscosity of mucus leads to build-up of large amplitude oscillations over multiple respiration cycles.

The processing unit is for example configured to track the integral of the power spectral density over successive breaths. This integral represents a measure of the oscillatory behavior. The integral may be limited to a certain spectral window, for instance 5-20 Hz, where the sensitivity is higher.

The processing unit may be configured to analyze an integral of the power spectral density of the ventilation waveform to determine an airway distribution of mucus.

Thus, the power spectral density may be used to determine a distribution as well as a viscosity. The oscillations observed mainly originate from mucus accumulation within the first generations (up to generation 2-3). Deeper mucus does not seem to contribute much to the observed fluctuations at the mouth or trachea. This difference in response, depending on the mucus depth, can thus be exploited to distinguish resistance increases due to mucus accumulation in the first generations from resistance increases due to deeper accumulation. The airway resistance may for example be monitored as an additional sensed characteristic. In both cases, the airway resistance increases. However, only when mucus is present in the first generations, the oscillatory behavior is observed. In this way, a mucus distribution can be classified as “deeply located” or “located at the first generations”.

The examples above relate to significant oscillations which are observed during the inspiratory phase of the operation waveform. However, the expiratory operation waveform (e.g. pressure trace) displays a modulation over time when thicker mucus is present. Thus, the processing unit may be configured to analyze the ventilation waveform during the expiratory phase to determine mucus thickness.

Such modulation may be used (for example in combination with the other parameters described above) to better identify changes in mucus thickness and to better distinguish them from e.g. flow related effects.

The processing unit is for example configured to take account of the patient physiological condition to assist in determining a mucus distribution.

Information from a patient record (e.g., EMR) may be used to improve the determination of the mucus distribution in the airway, i.e., the mucus build-up in the upper and lower airway. For example, patients with emphysema typically have build-up in the lower airway in generations 21-23. In contrast, COPD patients typically have mucus build-up in the upper airway, namely generations 0-5.

In summary, the examples above show that the mucus thickness may generally be derived from waveform features related to the amplitude and frequency of flow and/or pressure oscillations in the inspiratory and expiratory phases or alternatively by defining a single distinctive parameter based on the average or cumulative integral of the power spectral density (PSD) over a number of breaths. Mucus viscosity may be determined by analyzing the maximum PSD of the operation waveform over a number of successive breaths. Mucus airway distribution can be determined by looking at the oscillatory behavior and spectral effects in the integral of the PSD of the operation waveform.

The processing unit may be further adapted to monitor airway resistance and compare the airway resistance with a default value for the same ventilator flow to determine a change in mucus thickness. This may be for example based on an assumed mucus distribution.

The processing unit may be configured to derive a personalized non-pharmaceutical mucus loosening, thinning and clearance therapy.

For example, the personalization may comprise:

settings for a semi-automated mucus loosening, thinning or clearance therapy such as a frequency and/or duration of oscillations to be applied to the chest of a subject; or

a set of personalized ventilator settings such as a respiration frequency set by the ventilator, or a flow rate set by the ventilator or an inspiratory time set by the ventilator, or specific combinations of those.

The semi-automated mucus loosening, thinning or clearance therapy is for example an oscillating positive expiratory pressure therapy or a high frequency chest wall oscillation therapy.

For instance, if thicker and more viscous mucus is detected then these therapies may be performed for longer times and/or more frequently. Specific oscillation frequency ranges using oscillating positive expiratory pressure therapy may be adapted.

The inspiratory time and flow rate implemented by the ventilator may be adapted in order to maximize mucus clearance. Mucus clearance can be related to the net mucus volume within the system. In the context of this text, a positive value means that overall the mucus is being pushed downward towards the alveoli, while a negative value demonstrates that the mucus is being pushed upward toward the mouth to be cleared. An optimal inspiratory time and optimal range of inspiratory flows may be derived.

The invention also provides a patient ventilator system, comprising;

a patient ventilator;

a patient mask; and

the analysis system defined above, wherein the sensor system is for monitoring pressure and/or flow delivered to or from the patient mask.

The invention also provides a computer implemented method for analyzing physical characteristics of mucus during patient respiratory support using a ventilator, comprising:

receiving a sensed ventilation waveform;

analyzing the sensed ventilation waveform over time; and

deriving from the analysis an estimate at least one mucus characteristic.

The invention also provides a computer program comprising computer program code means which is adapted, when said program is run on a computer, to implement the method defined above.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1a shows a 0D representation of Weibel's symmetric airway tree used to provide the the airway tree dimensions as input and FIG. 1b shows a simplified 0D model of an arbitrary single airway (from the tree);

FIG. 2 shows a representation of the airway as modeled by a 3D numerical model;

FIG. 3 shows a pressure waveform for a thick mucus layer (plot 30) and for a thin mucus layer (plot 32) simulated from a 0D model;

FIG. 4 shows flow-volume curves;

FIG. 5 is a zoomed in view of the inspiratory part of FIG. 4;

FIG. 6 shows the integral of the power spectral density, PSD, (y-axis) of the pressure waveform tracked over a number of breaths (x-axis);

FIG. 7 shows a plot of pressure versus time for different mucus thickness;

FIG. 8 shows a PSD measurement (y-axis) over time (x-axis) for different mucus thickness for single breaths;

FIG. 9 shows a fast Fourier transform (y-axis) versus frequency (x-axis) for different mucus thickness;

FIG. 10 shows the maximum PSD for different mucus thickness across multiple consecutive breaths;

FIG. 11 shows corresponding PSDs values for different mucus thickness as averaged over multiple breaths;

FIG. 12 shows a table of mucus thickness for different generations for two example airway distributions;

FIG. 13 show the integral of PSD over a series of breaths for the two mucus distribution cases of FIG. 12;

FIG. 14 shows a pressure trace over time at the mouth and at the trachea;

FIG. 15 shows a PSD of a flow trace at the mouth and at the trachea;

FIG. 16 shows the effect of a ventilator flow rate on the power spectrum of the pressure signal for different respiratory rates (as well as the other input parameters);

FIG. 17 shows a plot of mucus thickness at the trachea (y-axis) versus resistance for different flow rates;

FIG. 18 shows a plot of a thickness variation ratio (i.e. a ratio between a current thickness and a thickness at the baseline) versus a resistance variation ratio (i.e. a ratio between a current resistance and a resistance at the baseline);

FIG. 19 shows a plot of net mucus volume (y-axis) versus mean inspiratory flow (x-axis) for a mucus dynamic viscosity of 0.1 Pa·s;

FIG. 20 shows a plot of net mucus volume (y-axis) versus mean inspiratory flow (x-axis) for a mucus dynamic viscosity of 10 Pa·s;

FIG. 21 is used to show the relationship between ventilator settings and mucus clearance and mucus properties (dynamic viscosity and thickness); and

FIG. 22 shows a system for analyzing physical characteristics of mucus during patient respiratory support using a ventilator.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a system for analyzing physical characteristics of mucus, in particular during patient respiratory support using a ventilator. A ventilation waveform is sensed and analyzed and an estimate of, or a change in, at least one mucus characteristic can then be determined.

The feasibility of using sensing of ventilation waveforms in order to derive characteristics of mucus (physical properties or distribution properties) has been determined by modeling the airway and analyzing the effect on the operation of ventilator based on different mucus properties and distributions.

There are various ways to model the airway.

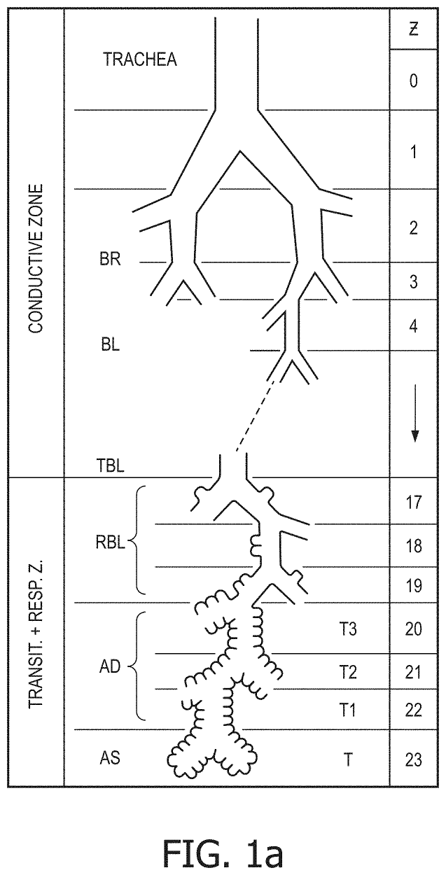

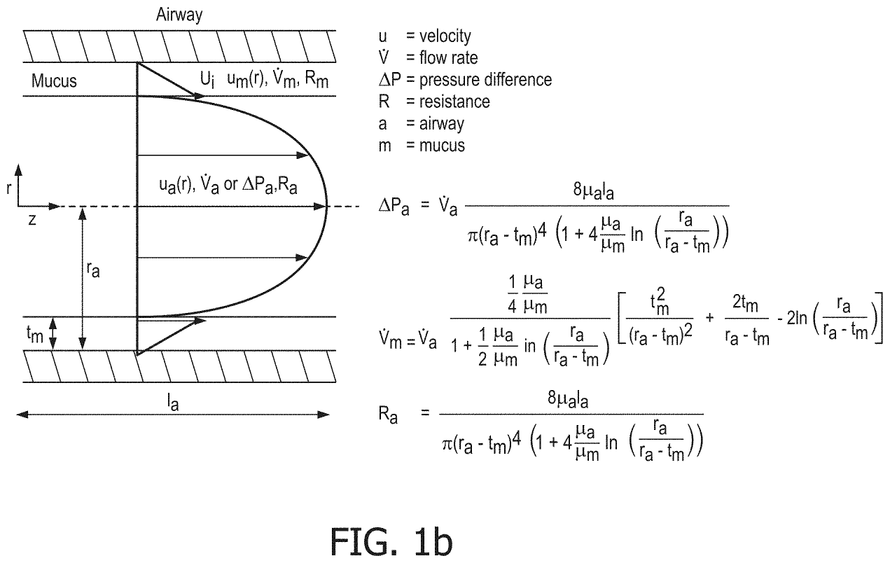

FIG. 1 shows the airway structure utilized for the 0D model based on Weibel's symmetric airway tree, as presented in Weibel ER. Morphometry of the Human Lung. Berlin Heidelberg: Springer-Verlag; 1963. The “Z” values denote the generation of airway tree, starting at 0 in the trachea. The airway structure of FIG. 1 is used to provide airway radius values as calculated by Weibel. These numbers are then used in the 0D model.

FIG. 1b shows the summary equations which form the basis of the 0D model. The airway radius rₐ is obtained from the Weibel airway tree.

By adding a mucus layer in the airways, a total airway resistance (considering the added mucus) is calculated. To model the full respiratory system, an additional respiratory compliance is added to the respiratory resistance, both of which form the basis of a lumped two-element (resistance & compliance) 0D model. The equations shown in FIG. 1b relate flow velocity, flow rate, pressure difference and resistance/compliance, which are applied at the set of locations of the modelled airway structure. Models such as this are known to those skilled in the art.

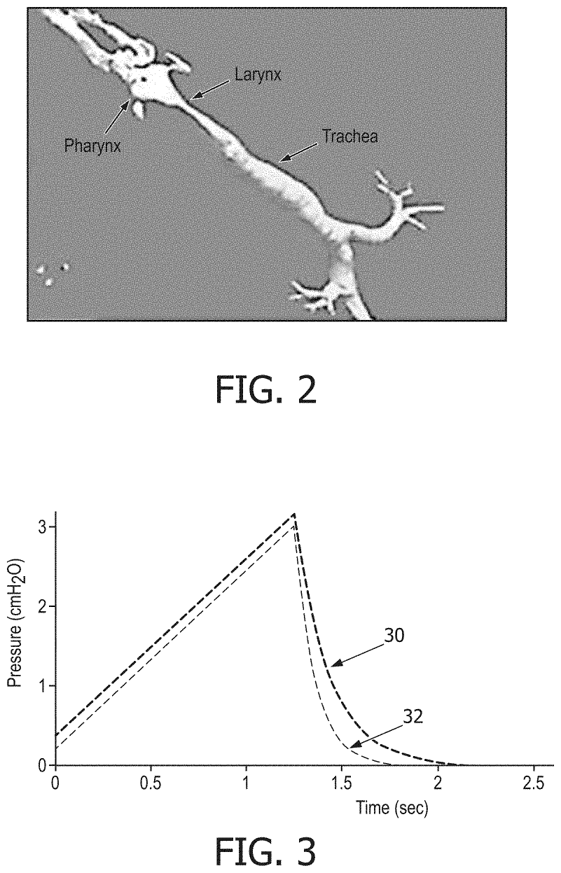

FIG. 2 shows a representation of the airway simulated by a numerical model, based on a realistic 3D geometry of the airway of an adult individual up to the 5^{th}/6^{th} generation.

The invention has been investigated using these two models.

From the 0D model (based on FIGS. 1a and 1b), flow (volume) and pressure waveforms were generated. For example, FIG. 3 shows a pressure waveform for a thick mucus layer (plot 30) and for a thin mucus layer (plot 32). The 0D model of FIG. 1 receives ventilator settings as an input, in order to create an inspiratory flow and volume (flow-integrated) waveform. The settings are for example body weight (in kg), tidal volume per ideal body weight (in ml/kg), flow profile, respiratory rate (breaths per minute, bpm) and expiratory to inspiratory ratio.

For respiratory mechanics, flow-dependent airway resistance and compliance are derived based on known techniques, such as described in Pedley T J, Schroter R C, Sudlow M F: “The prediction of pressure drop and variation of resistance within the human bronchial airways”, Respir Physiol 1970; 9:387-405.

The mucus thickness across the generations is set by predefining the mucus distribution profile (for example the mucus thickness varies proportionally to a change in airway diameter) and the thickness at the 0^{th} generation.

The waveforms are then used at the inflow boundary of the 3D model of FIG. 2. This is done for several reasons. The 0D model allows the simulation of flows and pressures across all generations 0 to 23. Thus, the 0D model may be used to generate data across the whole airway, whereas the 3D model is used for a few cases and only across the upper airways. It is not possible only to use the upper airways from the outset because the full airway needs to be modelled to derive the overall respiratory resistance and correct absolute pressure drop.

Thus, the two-model approach improves computational efficiency. The 0D model is very fast to run, but does not capture the full flow physics, whereas the 3D model is more accurate and is only applied to the region of interest of the airway to reduce computational processing requirements.

In order to provide a set of analysis data, the inputs were varied and simulated in a set of possible combinations, in particular resulting in 810 simulations used for the analysis.

The final model output is the inspiratory pressure waveform. Then the expiratory pressure, flow, and volumes are equated, hence assuming a passive expiration.

The model results were then utilized to investigate which ventilation waveform features are most useful in the characterization of mucus properties and the distribution of mucus in the airway.

A first application of the invention is to determine mucus physical properties and build-up in the airway based on analysis of ventilation waveforms. In particular, the invention enables changes in the in vivo mucus thickness, viscosity and/or airway distribution to be determined from features derived from the ventilation flow and/or pressure waveforms during a particular phase of the breathing cycle.

In use of a ventilator, a clinician can either control the flow or pressure delivered to the patient, but not both. Depending on the respiratory mechanics of the patient, the dependent variable (flow or pressure) is obtained as a sensed signal.

In the investigations performed, the flow was controlled by the ventilator, so the pressure is the sensed variable. The investigations were thus based on deciding a flow to deliver, and based on the mucus thickness, airway geometry, and lung compliance, a resulting pressure value was calculated using the model, thereby showing how the pressure is modulated by the different mucus characteristics. Thus, in a real system, the pressure would be monitored for analysis.

In use of the system, the pressure may instead be controlled and the flow could be monitored for analysis.

The pressure and flow are usually measured or estimated close to the mouth/airway of the patient. For example, a mask (known as a patient interface) can be connected to the ventilator through a long tube. The pressure and flow leaving the ventilator may be measured exactly and then with the known tube properties, the pressure can be estimated just before the mask (at the end of the tube). Furthermore, it is also possible that the patient is intubated rather than having a mask; but the idea is the same that the pressure (and flow) can be measured at the beginning or end of the tube.

### Mucus Thickness

The influence of mucus thickness has been investigated based on simulations using both the 0D and 3D models for mucus thicknesses ranging from 30 μm to 480 μm at the trachea (0^{th} generation) corresponding to normal/healthy individuals and for patients with pathological conditions.

To aid in analysis of the effects of mucus thickness, flow volume curves were explored in different simulations.

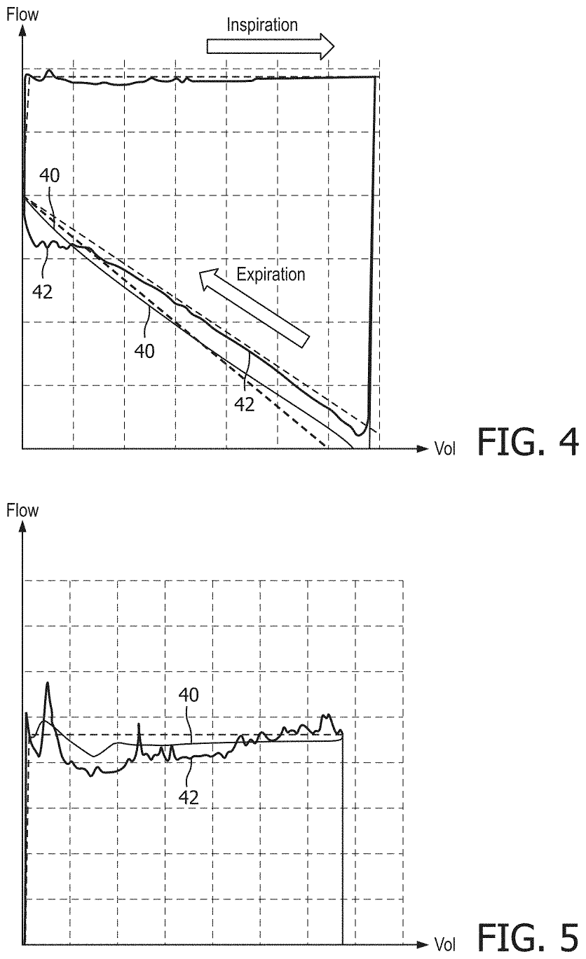

FIG. 4 shows flow-volume cyclic curves (plotting flow, y-axis, versus volume, x-axis). The inspiratory parts are at the top of the curve and expiratory parts are at the bottom. One cycle is shown as plot 40 for mucus thickness 30 μm at 10 bpm and 13 liters/minute and another cycle is shown as plot 42 for mucus thickness 480 μm at 20 bpm and 56 liters/minute.

FIG. 5 is a zoomed-in view of the inspiratory part of FIG. 4, to show more clearly the flow instabilities in the inspiratory phase which arise due to the presence of mucus of different thicknesses in the airway. Increasing flow instability is associated with thicker mucus.

From an analysis of different mucus thickness, different flow rates and different breath per minute (bpm) values, it becomes clear that at a given respiration rate, the flow instabilities are consistently more prominent when thicker mucus is present, particularly during the inspiration phase.

The analysis thus shows that at higher mucus thickness (e.g. 480 μm and 120 μm), prominent flow oscillations during the inspiratory phase are observed when compared to low mucus thickness (e.g. 30 μm). These oscillations are found to increase in amplitude and frequency with increasing mucus thickness at a constant respiration rate (RR). These oscillations are less prominent in the expiration phase.

The oscillations give rise to a larger spectral content of the flow-volume waveforms. The measurement of spectral content is for example applied to the ventilation pressure or the ventilation flow rate, or a combination of signals.

A possible way to detect automatically or semi-automatically the larger spectral content present in higher thickness cases is to look at the integral of the power spectral density (PSD) of the pressure waveform (in the case that the flow is controlled by the ventilator as explained above).

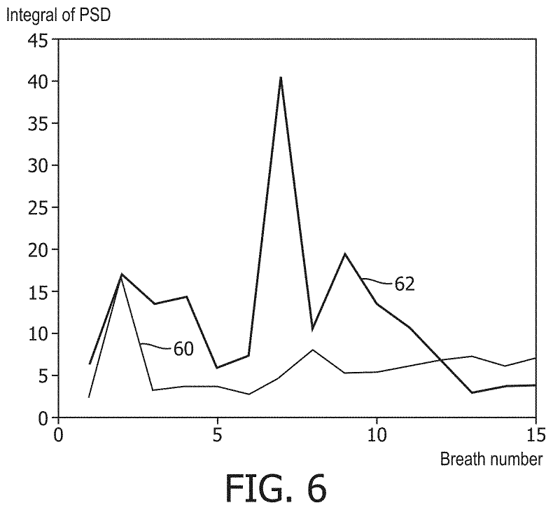

FIG. 6 shows the integral of the PSD (y-axis) of the pressure waveform tracked over a number of breaths (x-axis). Each plotted point is the PSD integral over one breath.

Plot 60 is for low mucus thickness and plot 62 is for high mucus thickness. The peak from breaths 6 to 8 arises due to the instabilities produced by the thicker mucus (located in particular in generations 0-3) in the inspiratory phase. These instabilities likely are caused by increased turbulence due to the narrowing of the airway and also air-mucus interfacial effects, since mucus is a viscoelastic non-Newtonian fluid.

Determining a maximum power spectral density over a number of successive breaths may thereby be used to determine a mucus viscosity.

The analysis could be further refined by limiting the integral to a certain spectral window, for instance 5-20 Hz, where the sensitivity of the method will be higher.

Also, a single distinctive parameter could be derived by averaging the curves in FIG. 6 over a number of breaths, or by studying a cumulative integral i.e. tracking an integral of the power spectral density over successive breaths. In both cases, a larger value of the derived parameter could be used as an indication of mucus build-up.

Thus, a determination of mucus thickness, or more particularly a change in mucus thickness, may be based on analyzing the ventilation waveform (pressure or volume flow rate) during the inspiration phase.

The method discussed above is based on the inspiration phase and on pressure-flow traces, where significant oscillations have been observed.

However, the expiratory pressure-flow characteristic typically also displays a modulation, for example in the pressure-time trace, when thicker mucus is present.

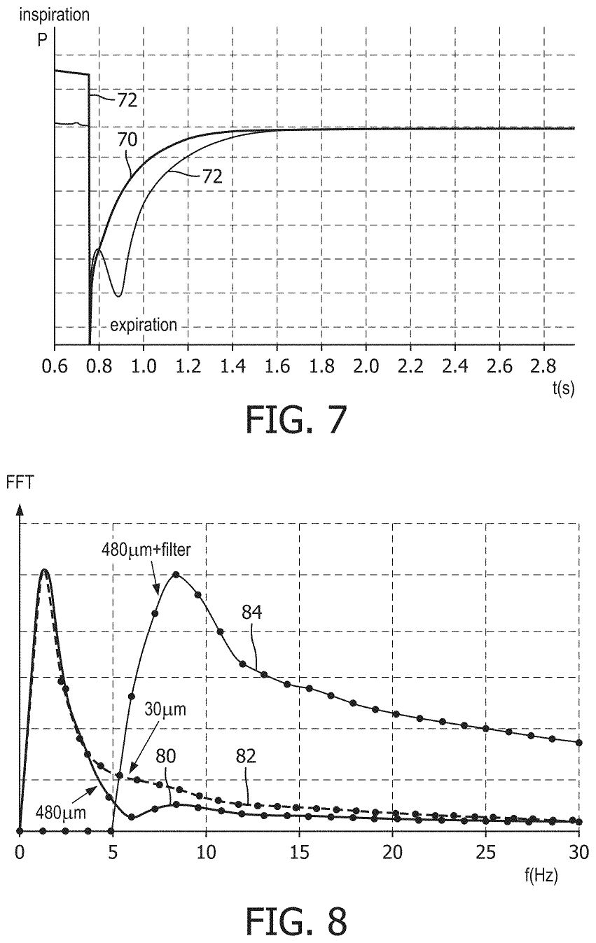

FIG. 7 shows a plot of pressure versus time. Plot 70 is for a thin mucus layer (30 μm) and plot 72 is for a thick mucus layer (480 μm). FIG. 7 shows that the effect of mucus thickness on the pressure trace modulation is particularly at the start of expiration. Plot 72 with the higher mucus thickness has higher pressure since the flow is controlled to be the same.

This pressure modulation of the expiration phase may be analyzed in combination with the variation of the ventilation waveform of the inspiration phase as discussed above. Changes in mucus thickness may thereby be identified and better distinguished from flow related effects.

FIG. 8 shows a PSD measurement (y-axis FFT value) for different frequencies (x-axis) for the expiration phase for a single breath.

Plot 80 is for thick mucus (480 μm), plot 82 is for thin mucus (30 μm) and plot 84 is for thick mucus with a post processing using a high pass filter in order to identify a modulation peak. FIG. 8 shows the modulation peak that arises due to the presence of thicker mucus, which is detected if the waveform is processed with a high pass filter.

### Mucus Viscosity

To investigate the effect of viscosity, two extreme cases may be considered: a normal ‘healthy’ case and a pathologic case. A higher viscosity (pathologic) case may be detected by looking at the trace of maximum PSD (e.g. the plot of FIG. 6) over a number of successive breaths.

A higher viscosity mucus leads to build-up of large amplitude oscillations over multiple respiration events. The appearance of a strong, low frequency (e.g. below 5-10 Hz) peak in the maximum PSD trace is an indication of more viscous mucus.

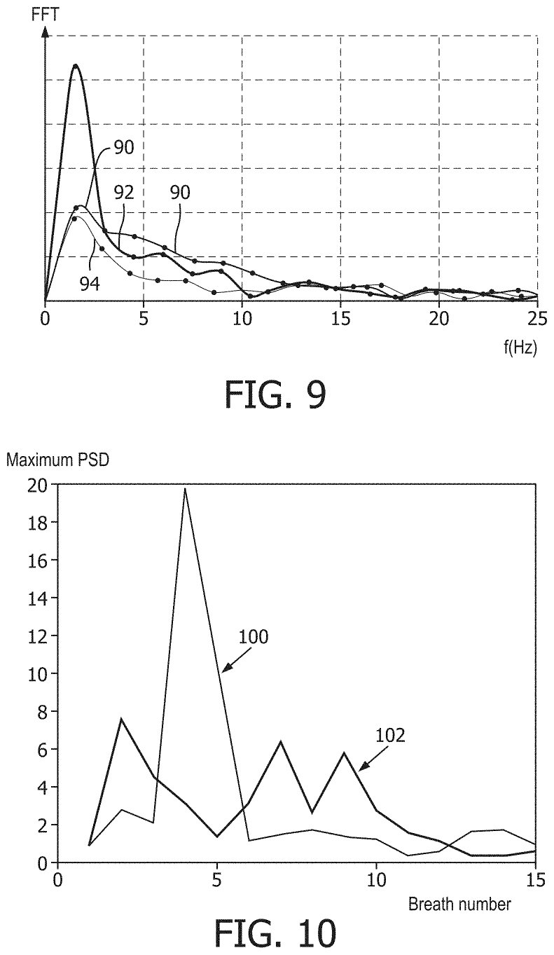

An example of results depicting this effect is shown in FIG. 9.

FIG. 9 shows the fast Fourier transform (y-axis) versus frequency (x-axis) and shows the oscillation build-up.

Plot 90 corresponds to the normal case, and plots 92 and 94 show the pathologic case at two different breaths.

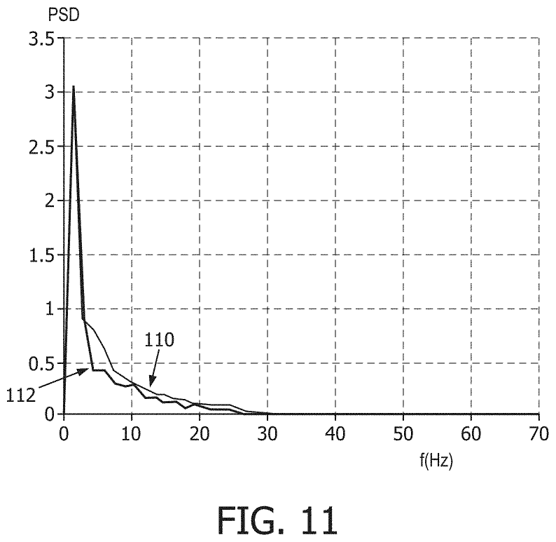

FIG. 10 shows the maximum PSD (plot 100 for high viscosity (pathological) mucus and plot 102 for normal viscosity mucus) and FIG. 11 shows corresponding PSDs values (plot 110 for high viscosity (pathological) mucus and plot 112 for normal viscosity mucus) over multiple breaths. The maximum and average Power Spectral Density (PSD) are shown over several successive breaths for two extreme cases of mucus viscosity, one normal (low viscosity) and one pathologic (high viscosity).

The difference between the maximum PSD for the normal and pathologic mucus cases is believed to be related to dynamic effects and/or to the non-Newtonian description of mucus.

The analysis of the power spectral density of the ventilation flow (pressure or volume flow rate) may thereby example enable a determination of mucus viscosity, or more particularly a change in mucus viscosity.

### Airway Distribution

The oscillations and related spectral effects mainly originate from mucus accumulation within the first generations (up to generation 2-3). Deeper mucus does not seem to contribute much to the observed fluctuations at the mouth or trachea.

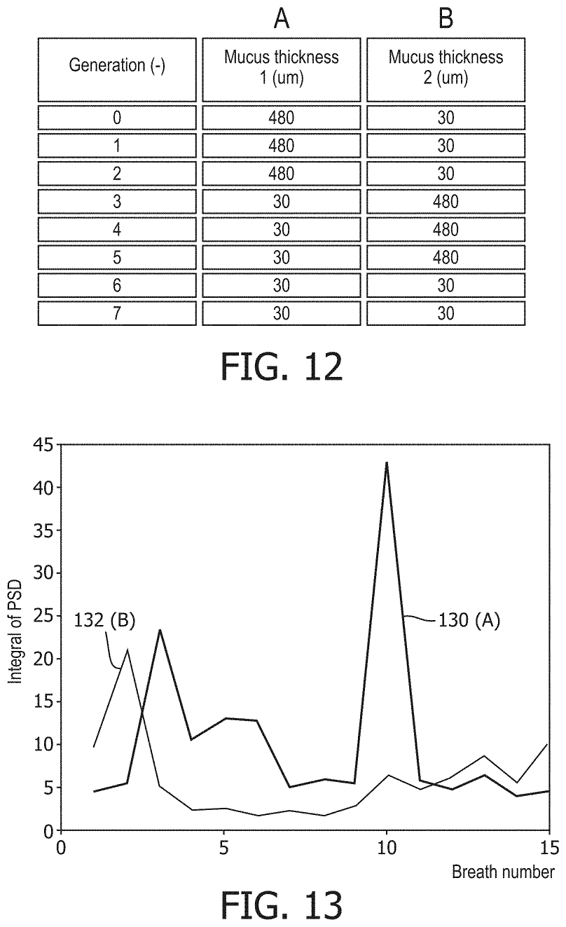

FIG. 12 shows a table of mucus thickness for different generations for two example airway distributions. Distribution A has thick mucus in the first generations 0 to 2 and Distribution B has thick mucus in generations 3 to 5.

FIG. 13 shows the integral of PSD (of the pressure signal) over a series of breaths for the two mucus distribution cases of FIG. 12. Plot 130 is for Distribution A and plot 132 is for Distribution B.

For Distribution B with deeper mucus, the response is qualitatively very close to a trace with 30 μm mucus thickness in all generations. This confirms that most of the observed oscillations are originated in the first few generation (0-3).

The differences in the PSD signal can be exploited to distinguish resistance increases due to mucus accumulation in the first generations from resistance increases due to deeper accumulation. In both cases, the airway resistance increases. However, only when mucus is present in the first generations, an oscillatory behavior is observed. This circumstance may be exploited to give the clinician or caregiver the possibility of classifying the mucus distribution as “deeply located” or “located at the first generations”.

Thus, the difference in the distribution of the mucus influences the integral of the PSD, since the effects of the instabilities caused by the presence of the thick mucus manifest themselves after a different number of breathing cycles. This is how the distribution of the mucus in the airway is determined.

### Additional Refinements

The estimation of mucus characteristics as explained above (physical properties and airway distribution) may be improved by accounting for the influence of the oral cavity.

In particular, frequency oscillations in the ventilation waveforms may be influenced by the oral cavity. Preliminary results on oral geometry, including mouth and nasal cavities, suggest that the effect on the pressure trace will be weak, implying that the recorded pressure trace should be a reasonably good surrogate for the trace at the trachea.

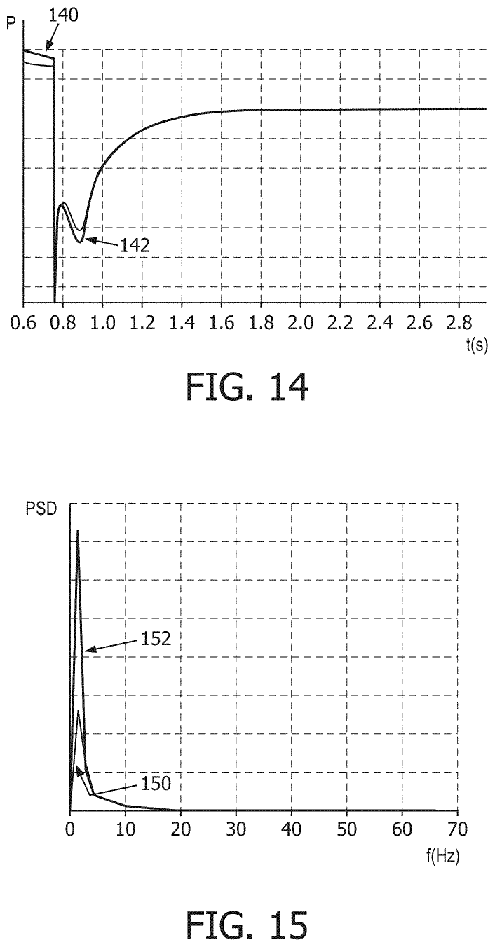

FIG. 14 shows a pressure trace over time at the mouth as plot 140 and at the trachea as plot 142.

FIG. 15 shows a PSD (averaged over multiple successive breaths) of a flow trace at the mouth as plot 150 and at the trachea as plot 152. The main effect of the oral geometry is a reduction of amplitude at the mouth, with little shift in spectral content.

Filtering and/or amplification of the signal may therefore be required to identify the relevant signal, typically amplification and filtering in the 5-20 Hz range. However, the signature of deeper airways is still detectable.

The estimation of mucus characteristics as explained above (physical properties and airway distribution) may also be improved by accounting for the influence of flow rate and patient type.

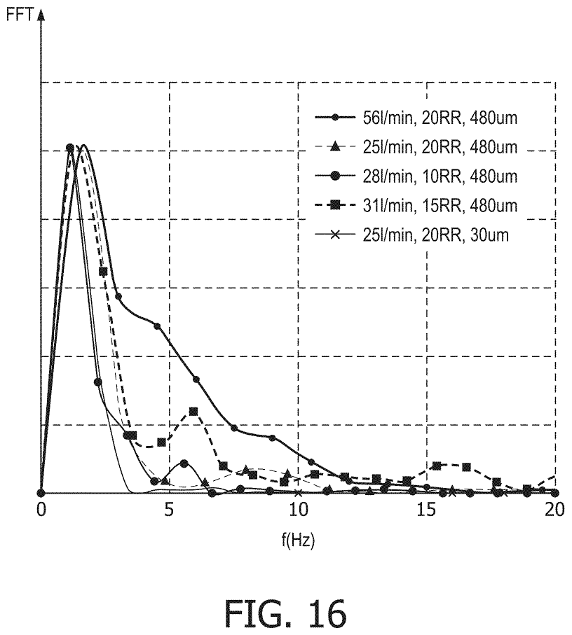

FIG. 16 shows the effect of the flow rate on power spectrum of the pressure signal for different respiratory rates. Five plots of the FFT power spectrum are shown for different combinations of flow rate (25 l/min, 28 l/min, 31 l/min and 56 l/min), respiration rates (10 bpm, 15 bpm and 20 bpm) and mucus thickness (30 μm and 480 μm).

As shown in FIG. 16, a higher flow rate will introduce stronger and more high frequency oscillations. This effect is not easily distinguished from the effect of mucus related spectral changes (particularly thickness).

Therefore, the methods described above may be considered as ways to detect a relative change of thickness or viscosity with respect to reference data, acquired at equal flows.

The analysis of inspiration spectral content could also be combined with the detection of modulation on the expiratory trace. This combined method will enable separation of the spectral content resulting from the flow rate from the mucus-related spectral content. However, the method is not intended to provide a quantification of an exact value of mucus thickness.

The determination of the location of the mucus build-up in the airway may be refined based on an input of the patient type or patient history, e.g. obtained from the electronic medical record (EMR) or other database containing the patient history.

For example, patients with emphysema typically have build-up in the lower airway in generations 21-23 generations. In contrast, COPD patients typically have mucus build-up in the upper airway, namely generations 0-5. As mentioned above, in some cases, mucus build-up (i.e., relative thickness) in the upper and lower airway may be hard to distinguish based solely on the ventilation flow waveform analysis, and this additional patient information enables more reliable evaluation.

In another approach, changes in mucus thickness may be determined based on airway resistance measurements at a given flow rate. A change (build-up or clearance) in mucus over time and/or resulting from treatment may be determined by monitoring the change in resistance from a baseline point. Ventilators are known which continuously and non-invasively measure resistance. The resistance can be compared at matching flow rates with a baseline point.

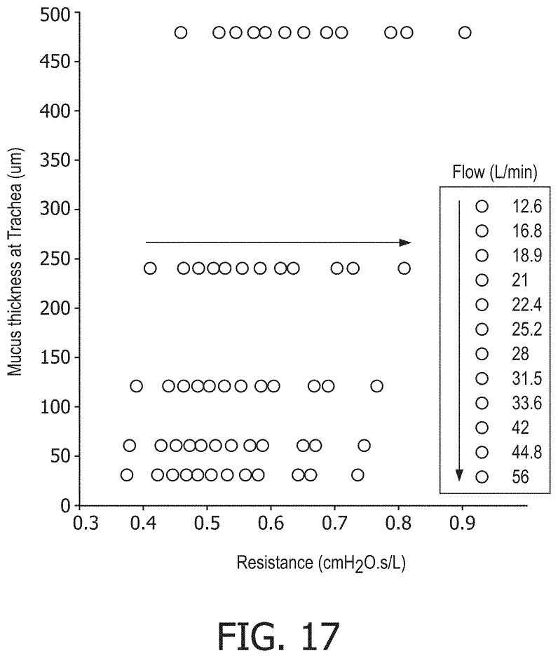

FIG. 17 shows a plot of mucus thickness at the trachea (y-axis) versus resistance for different flow rates. In each row of points in the graph (for a single value of mucus thickness) the flow increases from 12.6 l/min to 56 l/min. Thus, FIG. 17 shows that for different flow rates, the mucus thickness can be estimated from a given a resistance value at the specific flow rate, independent of compliance, mucus dynamic viscosity and all other input parameters.

In addition, a baseline could be chosen for every flow rate. In this way, the clinician can monitor how the change in resistance could explain a change in the mucus thickness across the airways.

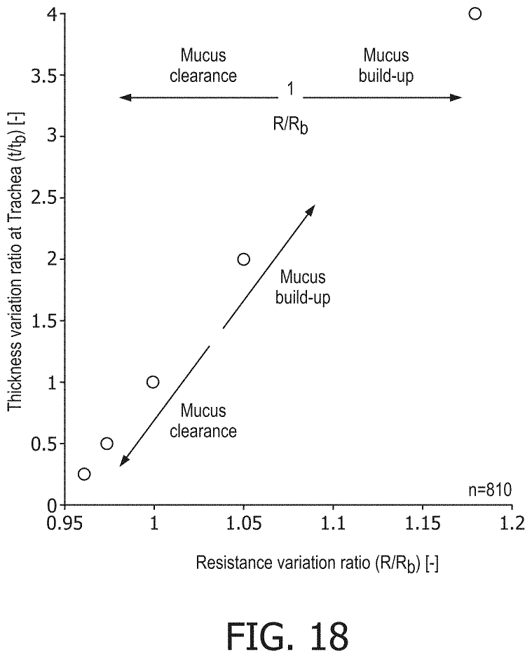

For example, FIG. 18 shows a plot of a thickness variation ratio (i.e. a ratio between a current thickness and a thickness at the baseline) versus a resistance variation ratio (i.e. a ratio between a current resistance and a resistance at the baseline) The absolute change in mucus thickness is dependent on mucus distribution, but the direction of change is independent of the distribution and can provide insight on clearance or build-up. In particular, the change in airway resistance correlates with the change in mucus thickness.

By combining a resistance value at a specific flow, the mucus thickness at the trachea can be estimated assuming a specific mucus distribution. Without knowledge of the mucus distribution, a generalized knowledge of the mucus build-up or clearance is achieved by measuring the resistance ratio. An increase in ratio at the same flow rate potentially signifies increase in mucus thickness and vice versa.

As mentioned above, one purpose of the determination of the mucus characteristics is to enable optimization and personalization of mucus loosening and clearance strategies.

The therapy may be a manual therapy, which is typically performed by a respiratory therapist doing chest percussion on the patient to loosen mucus which is then expelled by the patient.

Semi-automated therapies may be used such as Oscillating Positive Expiratory Pressure (OPEP) and High Frequency Chest Wall Oscillation (HFCWO). These are semi-automated in the sense that the patient still must expel the loosened mucus.

The mucus characteristics (viscosity, thickness and airway distribution) may be used to personalize and optimize mucus clearance therapy management. In the case of non-pharmaceutical clearance therapies such as manual therapy (i.e., chest percussion), OPEP and HFCWO, the duration and frequency of performing the therapy (e.g. oscillations applied to the chest by a vest) can be adapted.

For example, if thicker and more viscous mucus is detected, then HFCWO therapy may be performed for 20 minutes, 4 times per day instead of for 15 minutes, 3 times per day. In addition, the frequency of chest wall oscillation be adapted to improve the loosening of mucus. For example, for thicker more viscous mucus, it may be advantageous to user higher frequency oscillations to loosen the mucus. In yet another example, for mucus that is lodged in the lower airway it may be advantageous to apply oscillation to different compartments of a chest stimulation vest or to apply specific oscillation frequency ranges using OPEP.

Ventilator settings may also be adjusted. The inspiratory time and flow rate may also be adapted in order to maximize mucus clearance. Mucus clearance can be related to the net mucus volume within the system. A positive value means that overall the mucus is being pushed downward towards the alveoli, while a negative value demonstrates that the mucus is being pushed upward toward the mouth to be cleared.

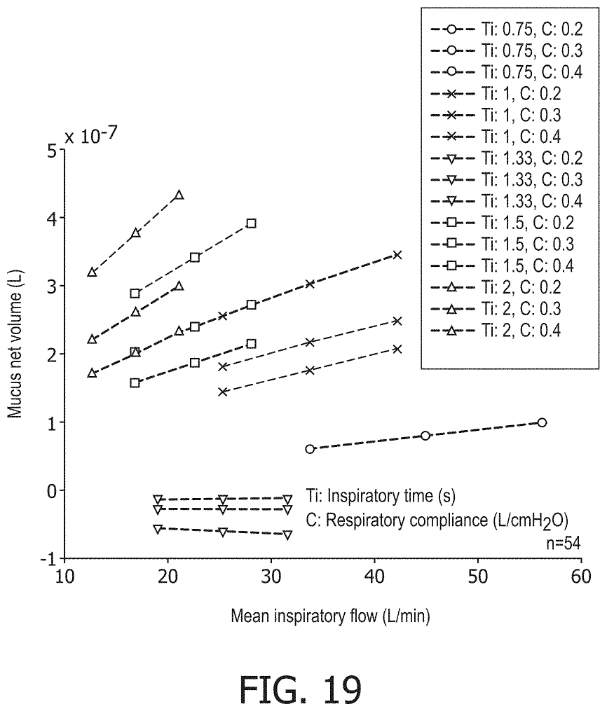

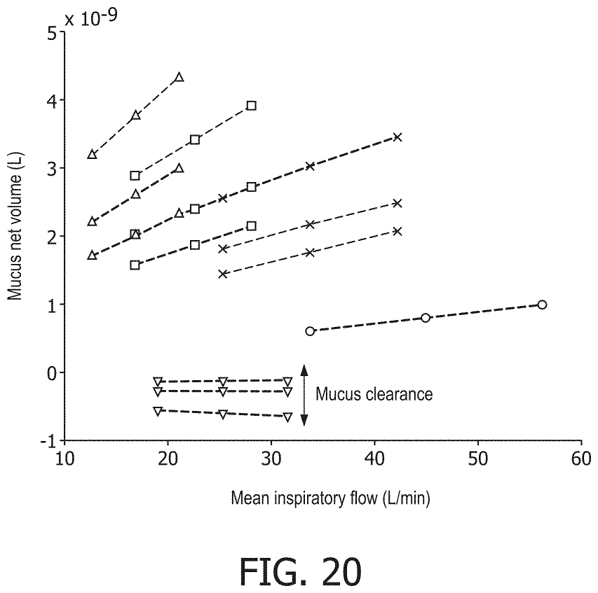

FIGS. 19 and 20 each show a plot of net mucus volume (y-axis) versus mean inspiratory flow (x-axis) for different combinations of inspiratory time and respiratory compliance, for 480 μm mucus thickness.

FIG. 19 is for a mucus dynamic viscosity of 0.1 Pa·s and FIG. 20 is for a mucus dynamic viscosity of 10 Pa·s.

The analysis enables the flow rate and inspiratory time to be optimized to maximize mucus clearance. For a mucus thickness of 480 μm at the trachea, mucus clearance occurs between the flow range of 18-32 l/min for an optimal inspiratory time of 1.33.

The increase in mucus dynamic viscosity by two order of magnitudes (from 0.1 to 10 Pa·s) does not affect these optimal settings but decreases the net value of the clearance with the same magnitude. In both cases, a decreased compliance increases the magnitude of clearance.

Thus, as FIGS. 19 and 20 demonstrate, there is an optimal inspiratory time and optimal range of inspiratory flows (between the maximum and minimum physiological values respectively) from which the net mucus volume is negative. These findings suggest that by monitoring the respiratory mechanics and the change in resistance, clinicians can change the ventilator waveforms to reduce this ratio and optimize mucus secretion and clearance.

The monitoring of resistance change and mucus clearance, as explained above, may be used to detect changes in mucus dynamic viscosity. In the 0D model, the resistance is independent of the mucus dynamic viscosity.

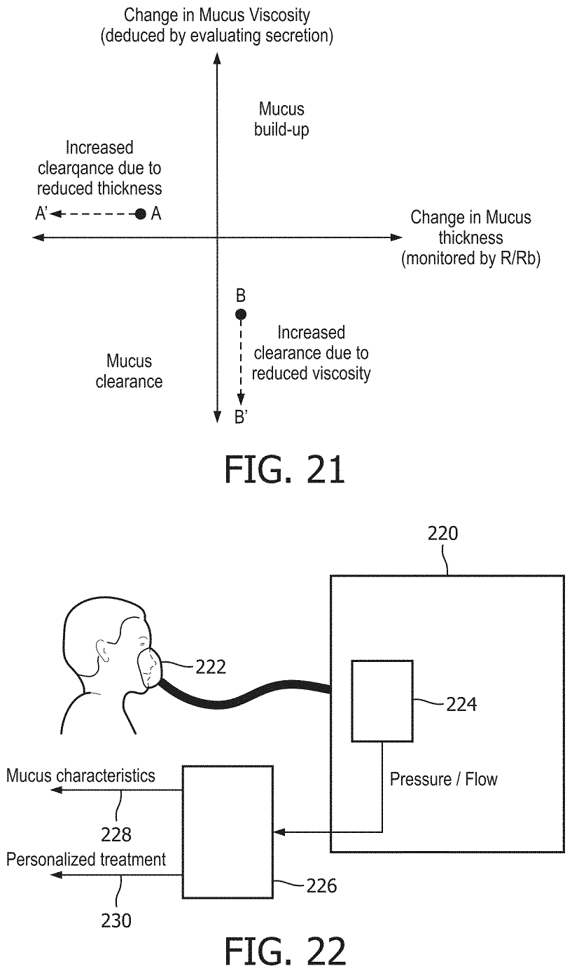

FIG. 21 is used to show the relationship between ventilator settings with mucus clearance and mucus properties (dynamic viscosity and thickness). Mucus clearance from the body is affected by the choice of ventilator settings and specifically flow and inspiratory time, and respiration rate. Improved mucus clearance occurs when there is reduced mucus thickness (A-A′) and when there is reduced dynamic viscosity (B-B′). The thickness can be monitored from the ratio of airway resistances as explained above. By observing the amount of secretions, changes in mucus viscosity could be captured. For example, increased clearance without a change in thickness suggests a decrease in dynamic viscosity such as from B-B′.

Thus, FIG. 21 shows that the effect of the dynamic viscosity resides in altering the magnitude of the clearance, and thus as the clearance increases with negligible changes in resistance (and thus mucus thickness) the dynamic viscosity can be monitored. In other words, by monitoring the change in resistance and secretions a clinician can have an insight into the change in mucus thickness and viscosity.

FIG. 22 shows a system for analyzing physical characteristics of mucus during patient respiratory support using a ventilator 220. The ventilator delivers breathing gas to a user via a patient interface 222, e.g. a mask.

The system comprises a sensor arrangement 224 for sensing a ventilation waveform. This may be part of a ventilator 220 or it may be a separate sensor system.

A processing unit 226 analyzes the sensed ventilation waveform over time and derives from the analysis an estimate of, or a change in, at least one mucus characteristic. This may be output as a mucus characteristics signal 228. Alternatively, or additionally, personalized treatment options may be provided as output 230.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”.

Any reference signs in the claims should not be construed as limiting the scope.

1. A system for analyzing physical characteristics of mucus during patient respiratory support using a ventilator, comprising:
a sensor arrangement for sensing a ventilation waveform;
a processing unit configured to:
analyze the sensed ventilation waveform over time; and
derive from the analysis an estimate of, or a change in, at least one mucus characteristic.

2. The system of claim 1, wherein the at least one mucus characteristic comprises one or more of:
a mucus thickness;
a mucus viscosity; and
a distribution of mucus in the airway of the patient.

3. The system of claim 1, wherein the sensed ventilation waveform comprises one or more of:
a ventilation pressure; and
a ventilation flow.

4. The system of claim 1, wherein the processing unit is configured to analyze a frequency spectrum content of the ventilation waveform.

5. The system of claim 1, wherein the processing unit is configured to analyze the ventilation waveform during an inspiration phase to determine mucus or a change in mucus thickness.

6. The system of claim 1, wherein the processing unit is configured to analyze a power spectral density of the ventilation waveform to determine a mucus viscosity or a change in mucus viscosity, for example wherein the processing unit is configured to:
determine a maximum power spectral density over a number of successive breaths; or
track the integral of the power spectral density over successive breaths.

7. The system of claim 1, wherein the processing unit is configured to analyze an integral of the power spectral density of the ventilation waveform to determine an airway distribution of mucus or a change in airway distribution of mucus.

8. The system of claim 1, wherein the processing unit is configured to analyze the ventilation waveform during the expiratory phase to determine a mucus thickness or a change in mucus thickness.

9. The system of claim 1, wherein the processing unit is configured to take account of a physiological condition of the patient to assist in determining a mucus distribution.

10. The system of claim 1, wherein the processing unit is further adapted to monitor airway resistance and compare the airway resistance with a default value for the same ventilator flow to determine a change in mucus thickness.

11. The system of claim 1, wherein the processing unit is configured to derive a personalized non-pharmaceutical mucus loosening, thinning and clearance therapy.

12. The system of claim 11, wherein the personalization comprises:
settings for a semi-automated mucus loosening, thinning or clearance therapy such as a frequency and/or duration of oscillations to be applied to the chest of a subject; and/or
a set of personalized ventilator settings such as a respiration frequency set by the ventilator, or a flow rate set by the ventilator or an inspiratory time set by the ventilator.

13. A patient ventilator system, comprising;
a patient ventilator;
a patient mask; and
the system of claim 1, wherein the sensor system is for monitoring pressure and/or flow delivered to or from the patient mask.

14. A computer implemented method for analyzing physical characteristics of mucus during patient respiratory support using a ventilator, comprising:
receiving a sensed ventilation waveform;
analyzing the sensed ventilation waveform over time; and
deriving from the analysis an estimate at least one mucus characteristic.

15. A computer program comprising computer program code means which is adapted, when said program is run on a computer, to implement the method of claim 14.
